(19) United States
(12) Patent Application Publication
(10) Pub. No.: US 2026/0335867 A1
(43) Pub. Date: Oct. 29, 2026

(54) HYDROTALCITE-BASED CATALYST FOR PRODUCING 1,2- HEXANEDIOL AND METHOD FOR PRODUCING 1,2-HEXANEDIOL IN PRESENCE OF CATALYST

(71) Applicant: ACTIVON CO., LTD., Cheongju-si (KR)

(72) Inventors: Pravin Pandharinath UPARE, Cheongju-si (KR); Hyung Sub SHIN, Cheongju-si (KR); Seung Hoon LEE, Wonju-si (KR); Youn Ki CHO, Yongin-si (KR)

(21) Appl. No.: 19/699,701

(22) Filed: Jun. 5, 2026

Related U.S. Application Data
(63) Continuation of application No. PCT/KR2025/008384, filed on Jun. 18, 2025.

(30) Foreign Application Priority Data
Jun. 19, 2024 (KR) 10-2024-0079646

(51) Int. Cl. B01J 23/00; B01J 6/00; B01J 23/62; B01J 23/835; B01J 37/00; B01J 37/03; B01J 37/16; C07C 29/141; C07C 31/20

(52) U.S. Cl. CPC B01J 23/007; B01J 6/001; B01J 23/626; B01J 23/835; B01J 37/009; B01J 37/031; B01J 37/16; C07C 29/141; C07C 31/20

(57) ABSTRACT

The present invention relates to a hydrotalcite-based catalyst for producing 1,2-hexanediol and a method for producing 1,2-hexanediol in the presence of the catalyst. More specifically, the present invention relates to: a catalyst having a specific combination of catalytically active metals supported on a hydrotalcite support, so as to convert methylfurfural, which is a reactant, into 1,2-hexanediol with high yield; and a method for producing 1,2-hexanediol using the catalyst.

### CROSS REFERENCE TO RELATED APPLICATIONS

This application is a Continuation of International Application No. PCT/KR2025-008334, filed on 2025 Jun. 18, which claims priority under 35 U.S.C. § 119(a) to Korean Patent Application No. 10-2024-0079646, filed on 2024 Jun. 19. The entire disclosures of the above-identified applications are hereby incorporated by reference herein in their entirety.

### TECHNICAL FIELD

The present disclosure relates to a hydrotalcite-based catalyst for producing 1,2-hexanediol and a method of producing 1,2-hexanediol in the presence of the catalyst. Specifically, the present disclosure relates to a catalyst for selectively producing 1,2-hexanediol from methylfurfural, which is a reactant, the catalyst employing hydrotalcite as a support and having a specific metal combination of catalytically active metals supported thereon, and to a method of producing 1,2-hexanediol in high yield using the catalyst.

### BACKGROUND ART

1,2-Hexanediol (C₆H₁₂O₂), which is a colorless water-soluble solid, is a high value-added compound having hydroxyl groups on the first and second carbon atoms adjacent to each other in the main chain having 6 carbon atoms in the structure, and is used in various fields, such as a raw material for polyesters and as a crosslinking agent in the polymer industry, or as a humectant, an antimicrobial agent, a preservative, and the like in the fields of cosmetics and personal care products.

Recently, a method of producing 1,2-hexanediol by reacting 1-hexene with hydrogen peroxide has been used (Patent Documents 1, 2, and 3). This method has the advantage that 1,2-hexanediol can be produced economically and in high yield using hydrogen peroxide, which is an inexpensive oxidizing agent.

Although the aforementioned 1-hexene is readily available as a compound primarily obtained from petrochemical raw materials, which is advantageous, there is a growing demand for research and development of a technology for producing 1,2-hexanediol using biomass platform compounds as raw materials instead of petrochemical raw materials, under the recent trend of concerns over climate change and the strengthening of carbon neutrality policies. In one example, a method of producing 1,2-pentanediol from biomass-derived furfural as a raw material in the presence of an Rh/OMS-2 catalyst has been reported (Non-Patent Document 1).

Currently, a method of producing 1,2-hexanediol through a hydrogenation reaction of biomass-derived methylfurfural is being studied and developed as an important technology capable of utilizing biomass, and catalysts therefor are under development. However, there has been no report on the development of a novel catalyst with higher reaction selectivity for 1,2-hexanediol in the above method, as well as on a method of producing 1,2-hexanediol using the same. Thus, technological development in this regard is required.

The inventors of the present disclosure have conducted studies in search of a catalyst system capable of producing 1,2-hexanediol in high yield from a biomass-derived raw material, and, as a result, confirmed that the reaction selectivity for 1,2-hexanediol increases when a catalyst system including a specific metal combination of catalytically active metals and a hydrotalcite support is used, thereby completing the present disclosure.

### DISCLOSURE

### Technical Problem

The present disclosure aims to provide a hydrotalcite-based catalyst capable of producing 1,2-hexanediol in high yield using biomass-derived methylfurfural as a reactant, and a method of producing 1,2-hexanediol in the presence of the catalyst.

### Technical Solution

To solve the above problem, a catalyst of the present disclosure used for producing 1,2-hexanediol through a reaction between methylfurfural and hydrogen is characterized by having catalytically active metals supported on a hydrotalcite of Formula 1 below,

[Mg₁₋ₓAlₓ(OH)₂][(CO₃²⁻)_{x/2}]·nH₂O [Formula1]
(wherein in Formula 1, x is 0.2×0.33, and n is 0.33≤n≤1),
wherein the catalytically active metals include: tin (Sn); and a metal selected from the group consisting of ruthenium and nickel.

In one embodiment of the present disclosure, methylfurfural may be biomass-derived.

An amount of tin supported on the catalyst may be 20 wt % or less, preferably 10 wt % or less, more preferably 5 wt % or less, and most preferably in the range of 1 to 5 wt %.

In one embodiment of the present disclosure, the catalyst may be used by reduction without calcination in an oxidizing atmosphere.

In addition, the present disclosure may provide a method of producing 1,2-hexanediol in the presence of the hydrotalcite-based catalyst.

The method of producing 1,2-hexanediol may be characterized in that 1,2-hexanediol is produced through a liquid-phase reaction between methylfurfural and hydrogen in the presence of a hydrotalcite-based catalyst for producing 1,2-hexanediol, wherein methylfurfural is diluted in any one solvent selected from 1-propanol, isopropanol, 1-butanol, 2-butanol, and a mixture thereof, and subjected to the liquid-phase reaction.

The reaction in the presence of the hydrotalcite-based catalyst may be characterized by being performed at a temperature in the range of 120 °C to 200 °C, which is preferably in the range of 120 °C to 140 °C, and at a pressure in the range of 20 to 40 bar, which is preferably in the range of 20 to 30 bar.

### Advantageous Effects

According to the present disclosure, when a catalyst system including a specific metal combination of catalytically active metals and a hydrotalcite support is used, biomass-derived methylfurfural is used as a reactant, and 1,2-hexanediol can be produced in high yield.

### BEST MODE

Unless defined otherwise, all terms including technical and scientific terms used herein have the same meaning as commonly understood by those skilled in the art to which the present disclosure pertains. In general, the nomenclature used herein is well-known and commonly used in the art.

Throughout this specification, when a part is referred to as “comprising” or “including” a component, this means that other components may be further included, but does not preclude the presence thereof unless the context clearly indicates otherwise.

Hereinafter, a hydrotalcite-based catalyst for producing 1,2-hexanediol and a method of producing 1,2-hexanediol in the presence of the catalyst according to the present disclosure are to be described.

The catalyst used for producing 1,2-hexanediol through a reaction between methylfurfural and hydrogen according to the present disclosure is characterized by having a specific combination of catalytically active metals supported on a hydrotalcite of Formula 1 below,

[Mg₁₋ₓAlₓ(OH)₂][(CO₃²⁻)_{x/2}]·nH₂O [Formula1]
(wherein in Formula 1, x is 0.2≤x≤0.33, and n is 0.33≤n≤1),
wherein the catalytically active metals include: tin (Sn); and a metal selected from the group consisting of ruthenium and nickel.

Methylfurfural may undergo a one-step direct hydrogenation reaction, thereby producing 1,2-hexanediol and a mixture including the same, and the reaction yield of 1,2-hexanediol may be controlled depending on the activity of catalyst components used.

Methylfurfural may be a biomass-derived compound, specifically derived from hemicellulose. Xylan, which constitutes most of hemicellulose, is hydrolyzed to xylose, a pentose, and the resulting xylose may be dehydrated to readily obtain furfural.

An amount of tin supported on the catalyst may be 20 wt % or less, preferably 10 wt % or less, more preferably 5 wt % or less, and most preferably in the range of 1 to 5 wt %.

The catalytically active metals are supported on a support and preferably form a heterogeneous catalyst system. The present disclosure is characterized in that a hydrotalcite having Formula 1 below is used as the support.

[Mg₁₋ₓAlₓ(OH)₂][(CO₃²⁻)_{x/2}]·nH₂O [Formula1]

(In Formula 1, x is 0.2≤x≤0.33, and n is 0.33≤n≤1.)

The catalytically active metals may be supported on the hydrotalcite support in an amount in the range of 8 to 12 wt %, which is preferably in the range of 9 to 11 wt %. When the loading of the catalytically active metals falls within the above range, an optimal effect with respect to both catalytic activity and loading is exhibited.

A method of preparing the catalyst system in which the catalytically active metals are supported on a hydrotalcite support may include pre-forming a hydrotalcite and supporting precursors of the active metals by an impregnation method or the like, or may include supporting the active metals by a co-precipitation method in which a material used for preparing a hydrotalcite, as well as precursors of the catalytically active metals, are co-precipitated.

The precursor of tin serving as the catalytically active metal is not particularly limited in terms of type, and, for example, one or more components selected from Sn(NO₃)₂, SnCl₂, SnBr₂, SnI₂, Sn(OH)₂, SnSO₄, Sn(CH₃COO)₂, Sn(CH₃COCHCOCH₃)₂, SnO, SnO₂, and Sn₃(PO)₄ may be used.

The precursors of ruthenium and nickel serving as the catalytically active metals are not particularly limited in terms of type, and, for example, one or more selected from halides, nitrates, carbonates, sulfates, nitrosyl acetates, nitrosyl nitrates, and the like may be used.

In the meantime, the present disclosure may provide a method of preparing the catalyst for producing 1,2-hexanediol. The method of preparing the catalyst for producing 1,2-hexanediol, the catalyst having catalytically active metals including tin (Sn) and a metal selected from the group consisting of ruthenium and nickel supported on the hydrotalcite of Formula 1, includes the following steps: a) dispersing the hydrotalcite support in water or an organic solvent to prepare a hydrotalcite support dispersion; b) adding precursors of active components including a tin precursor and a precursor selected from the group consisting of a ruthenium precursor and a nickel precursor to the hydrotalcite support dispersion, and then stirring the resulting solution; c) drying the solution obtained after the stirring in Step b) to obtain a dried product; d) calcining the dried product to obtain a calcined product; e) reducing the calcined product to obtain a catalyst for producing 1,2-hexanediol, the catalyst having tin (Sn) and a metal selected from the group consisting of ruthenium and nickel as catalytically active metals supported on the hydrotalcite of Formula 1.

In one embodiment of the present disclosure, the order of adding the hydrotalcite support and the catalytically active metals may be changed such that Step a) is changed to Step a′) of preparing a solution in which precursors of catalytically active metals including a tin precursor and a precursor selected from the group consisting of a ruthenium precursor and a nickel precursor are dissolved, and Step b) is changed to Step b′) of dispersing the hydrotalcite support of Formula 1 prepared in advance in the solution in which the tin precursor and the precursor selected from the group consisting of the ruthenium precursor and the nickel precursor are dissolved.

In addition, in another embodiment of the present disclosure, Steps a) and b) may be performed simultaneously, such that the support and the precursors of the active metal components are added simultaneously.

In the meantime, in one embodiment of the present disclosure, the precursors in Step b) or b′) may be directly added to the solution of Step a) or a′). Alternatively, the precursors in Step b) or b′) may be separately dissolved in respective solvents, and the resulting solutions may be individually added to the solution of Step a) or a′).

Additionally, in another embodiment of the present disclosure, the calcination in Step d) may be omitted, and the process may proceed directly to Step e).

Furthermore, the method of preparing the catalyst for producing 1,2-hexanediol, according to the present disclosure, may be implemented not only by the aforementioned impregnation method but also by a co-precipitation method. In another embodiment, the method of preparing the catalyst according to the present disclosure includes the following steps: i) measuring predetermined amounts of a precursor used for preparing the hydrotalcite of Formula 1 and precursors of catalytically active metal components including a tin precursor and a precursor selected from the group consisting of a ruthenium precursor and a nickel precursor, and dissolving the precursors in a solvent to prepare a solution containing the support and the precursors of the catalytically active metals; ii) adjusting the pH of the solution containing the support and the precursors of the catalytically active metals; iii) subjecting the solution, the pH of which has been adjusted in Step ii), to aging at a predetermined temperature; iv) filtering a precipitate from the solution, which has been aged in Step iii), optionally washing the precipitate, and drying the precipitate; v) calcining the dried precipitate; vi) reducing the calcined product.

In one embodiment of the present disclosure, in Step i), the precursor used for preparing the hydrotalcite of Formula 1 and the precursors of the catalytically active metals including the tin precursor and the precursor selected from the group consisting of the ruthenium precursor and the nickel precursor may be simultaneously added to the solvent. Alternatively, solutions prepared by dissolving the respective precursors separately may be mixed to prepare the solution containing the support and the precursors of the catalytically active metals. In this case, as for the mixing order, the precursors of the catalytically active metals including the tin precursor and the precursor selected from the group consisting of the ruthenium precursor and the nickel precursor may be first mixed, followed by mixing the precursor used for preparing the hydrotalcite of Formula 1, or the reverse order may be employed.

In addition, in another embodiment of the present disclosure, the precursor used for preparing the hydrotalcite of Formula 1 and the precursor selected from the group consisting of the ruthenium precursor and the nickel precursor may be first mixed, followed by mixing the tin precursor. Alternatively, the precursor used for preparing the hydrotalcite of Formula 1 and the tin precursor may be first mixed, followed by mixing the precursor selected from the group consisting of the ruthenium precursor and the nickel precursor. Other variations in mixing order are also possible. However, when Step i) is not a single step but is performed as a sequential mixing process, the pH adjustment in Step ii) is performed after the sequential mixing, and the aging in Step iii) may be carried out after the final mixing or after each mixing step.

In the meantime, in another embodiment of the present disclosure, the calcination step v) may be omitted, and the process may proceed directly to the reduction step vi).

The aging in Step iii) may be carried out by allowing the solution to stand still at an elevated temperature or by allowing the solution to stand still without additional heating.

The pH adjustment in Step ii) may be performed using a basic substance and an acidic substance, and NaOH is preferably used.

In addition, the reduction step vi) may include subjecting the calcined product to a liquid-phase reduction process using a reducing agent such as hydrazine or NaBH₄, or to a heat treatment process in a hydrogen atmosphere. This is for controlling the dispersion and specific surface area of the catalytically active metals, removing impurities of the catalyst itself, and enhancing bonding strength between the catalytically active metals and the support. Preferably, the calcined product is subjected to the reduction at a temperature in the range of room temperature to 500 °C.

In the reduction step vi), the metals present as the catalytically active metals may be entirely reduced, or only partially reduced. For example, when ruthenium and tin are used as the active metals, ruthenium may be present in a reduced metallic state, whereas tin may partially remain unreduced and exist in a state combined with oxygen or the like, such as Sn²⁺ or Sn⁴⁺. In addition, the catalytically active metals including tin and the metal selected from the group consisting of ruthenium and nickel may form an alloy with one another.

In addition, the method of producing 1,2-hexanediol according to the present disclosure is characterized in that 1,2-hexanediol is produced through a reaction between methylfurfural and hydrogen in the presence of a catalyst having catalytically active metals including tin and a metal selected from the group consisting of ruthenium and nickel supported on a hydrotalcite support.

Methylfurfural may be a biomass-derived compound, specifically derived from hemicellulose. Xylan, which constitutes most of hemicellulose, is hydrolyzed to xylose, a pentose, and the resulting xylose may be dehydrated to readily obtain methylfurfural.

The amount of the hydrotalcite-based catalyst used for producing 1,2-hexanediol is preferably in the range of 1 to 15 wt % with respect to methylfurfural. When the amount of the catalyst is less than 1 wt %, the effect of activating the catalyst may be insufficiently exhibited. When the amount of the catalyst exceeds 15 wt %, it may be uneconomical in terms of the effect of increasing the catalytic activity relative to the amount of the catalyst.

The reaction in the presence of the catalyst may be carried out in a liquid phase or a gas phase, and is preferably carried out in a liquid phase.

For example, the reaction in the presence of the catalyst may be performed at a temperature in the range of 120 °C to 200 °C, which is preferably in the range of 120 °C to 140 °C, and at a hydrogen pressure in the range of 20 to 40 bar, which is preferably in the range of 20 to 30 bar. When the hydrogen pressure is less than 20 bar, the reaction rate may be slow, which is problematic. When the hydrogen pressure exceeds 40 bar, the reaction yield of by-products may increase, thereby decreasing the reaction yield of 1,2-hexanediol, which is problematic.

The reaction of the present disclosure may be carried out in a liquid phase. In this case, the reaction is carried out under conditions in which methylfurfural is maintained in a liquid state.

The methylfurfural may be diluted in a solvent capable of dissolving methylfurfural and used, and the solvent is preferably included in an amount in the range of 25 to 1000 wt % with respect to methylfurfural. Non-limiting examples of the solvent may include alcohols, gamma-butyrolactone (GBL), water, and mixtures thereof. Preferably, the solvent is an alcohol and is more preferably 1-propanol, isopropanol, 1-butanol, 2-butanol, or a mixture thereof.

The alcohol solvent has high solubility for methylfurfural, does not include highly reactive sites, and thus does not involve changes in functional groups or drastic changes in chemical properties as a result of the reaction with hydrogen. Accordingly, stable reaction conditions during the reaction process may be provided.

When the reaction between methylfurfural and hydrogen is carried out using the catalyst having the catalytically active metals including tin (Sn) and the metal selected from the group consisting of ruthenium and nickel supported on the hydrotalcite support of Formula 1 according to the present disclosure, the reaction yield of 1,2-hexanediol increases.

The reaction yield of 1,2-hexanediol is preferably 75% or more, more preferably 85% or more, and most preferably 90% or more.

Hereinafter, preferred embodiments of the present disclosure are to be described. It should be noted that the following examples are provided to illustrate one or more preferred embodiments of the present disclosure, and the present disclosure is not limited to these embodiments. Various modifications may be made to the following examples within the scope of the present disclosure.

### Example 1: Preparation of 10 wt % 1Ni-1Sn/Hydrotalcite Catalyst

Hydrotalcite was purchased from Sigma-Aldrich for use. Ni(NO₃)₂·6H₂O and SnCl₄·5H₂O were weighed in predetermined amounts such that the molar ratio of Ni to Sn was 1:1 and the total mass of Ni and Sn was 10 wt %, and were supported on the hydrotalcite by an impregnation method, thereby obtaining 1Ni-1Sn/Hydrotalcite.

### Example 2: Preparation of 4.66 wt % 1Ru-1Sn/Hydrotalcite Catalyst

1Ru-1Sn/Hydrotalcite was prepared in the same manner as in Example 1, except that RuCl₃ was used instead of Ni(NO₃)₂·6H₂O.

### Comparative Example 1: Preparation of 10 wt % 1Ni-1Sn/ZnO Catalyst

1Ni-1Sn/ZnO was prepared in the same manner as in Example 1, except that ZnO was used instead of the hydrotalcite.

### Comparative Example 2: Preparation of 4.66 wt % 1Ru-1Sn/ZnO Catalyst

1Ru-1Sn/ZnO was prepared in the same manner as in Comparative Example 1, except that RuCl₃ was used instead of Ni(NO₃)₂·6H₂O.

### Experimental Example: Production of 1,2-Hexanediol Using Catalysts of Examples and Comparative Examples

A 20 mL batch reactor system equipped with a temperature controller, a pressure controller, and a magnetic stirrer was prepared. Then, 1.5 g (10 wt % based on the feed) of the hydrotalcite-based catalyst of Example 1 or 2 or the ZnO-based catalyst of Comparative Example 1 or 2, which had been reduced at 500 °C for 6 hours before the reaction, was added to the reactor containing 15 g of a feed (isopropyl alcohol (IPA) solvent) having a methylfurfural (MF) concentration of 10%. The reactor system was sealed and heated to a temperature in the range of 120 °C to 140 °C at an H₂ pressure in the range of 20 to 30 bar. The reaction was initiated with stirring at 500 rpm. When the MF peak disappeared, completion of the reaction was confirmed, and the yield of the product obtained as a result of the reaction was measured by gas chromatography (GC). The results thereof are shown in Table 1 below.

TABLE 1

| | H₂ | | | | Yield (%) | | | |
|---|---|---|---|---|---|---|---|---|
| | Pressure | Temperature | Time | Conversion | | 1,2- | 1,5- | |
| Catalyst | (bar) | (°C) | (hr) | Rate (%) | DMF* | HDO** | HDO*** | Others**** |
| Example 1 | 20 | 120 | 72 | 100 | 5.0 | 82.1 | 5.8 | 7.1 |
| (Ni—Sn/HT) | | 130 | 59 | 100 | 5.2 | 82.6 | 5.1 | 7.1 |
| | | 140 | 48 | 100 | 5.5 | 81.5 | 5.5 | 7.5 |
| | 30 | 130 | 53 | 100 | 3.6 | 83.2 | 5.7 | 7.5 |
| Example 2 | 30 | 130 | 41 | 100 | 5.9 | 81.7 | 4.8 | 8.7 |
| (Ru—Sn/HT) | | | | | | | | |
| Comparative | 30 | 130 | 57 | 100 | 6.1 | 75.6 | 8.3 | 10.0 |
| Example 1 | | | | | | | | |
| (Ni—Sn/ZnO) | | | | | | | | |
| Comparative | 30 | 130 | 43 | 100 | 8.5 | 75.3 | 6.5 | 9.7 |
| Example 2 | | | | | | | | |
| (Ru—Sn/ZnO) | | | | | | | | |

*DMF: dimethylfuran,
**1,2-HDO: 1,2-hexanediol,
***1,5-HDO: 1,5-hexanediol,
****Others: oligomers + unclear components

Referring to Table 1, as a result of performing the methylfurfural conversion reaction in the presence of the Ni—Sn/Hydrotalcite catalyst of Example 1, under a constant pressure condition at 20 bar, the highest 1,2-HDO yield was obtained when the reaction was carried out at a temperature of 130 °C. In addition, as a result of performing the methylfurfural conversion reaction in the presence of the Ni—Sn/Hydrotalcite catalyst of Example 1 while increasing the pressure to 30 bar, the time required for completion of the reaction was shortened by 11%, and an equivalent or higher level of the yield was obtained (an increase of 0.6% p).

In the meantime, to compare catalytic activities according to the type of hydrotalcite support, conversion reactions were performed in the presence of any one of the catalysts of Examples 1 and 2 and Comparative Examples 1 and 2 under the following constant conditions: 130 °C and 30 bar. As a result, the catalysts of Examples 1 and 2, employing the hydrotalcite support, showed higher 1,2-HDO yields than those of Comparative Examples 1 and 2, employing a conventional ZnO-based support. In addition, the catalyst of Example 1, employing Ni—Sn as the active metals, exhibited a much higher 1,2-HDO yield than the catalyst of Example 2, employing Ru—Sn.

Furthermore, under the above reaction conditions, the H₂ pressure was fixed constantly at 20 bar, and the temperature range was expanded to 120 °C to 200 °C, after which the reaction was performed. The GC measurement results are shown in Table 2 below.

TABLE 2

| | H₂ | | | | Yield (%) | | | | |
|---|---|---|---|---|---|---|---|---|---|
| | Pressure | Temperature | Time | Conversion | | 1,2- | 1,5- | | |
| Catalyst | (bar) | (°C) | (hr) | Rate (%) | DMF* | HDO** | HDO*** | Others**** | Reactor |
| Example 1 | 20 | 120 | 72 | 100 | 5.0 | 82.1 | 5.8 | 7.1 | 20 mL |
| (Ni—Sn/HT) | | 130 | 59 | 100 | 5.2 | 82.6 | 5.1 | 7.1 | |
| | | 140 | 48 | 100 | 5.5 | 81.5 | 5.5 | 7.5 | |
| | | 150 | 44 | 100 | 7.2 | 78.3 | 5.7 | 8.8 | |
| | | 160 | 38 | 100 | 6.9 | 75.2 | 6.3 | 11.6 | |
| | | 180 | 31 | 100 | 5.9 | 73.3 | 8.2 | 12.6 | |
| | | 200 | 30 | 100 | 8.3 | 65.03 | 7.8 | 18.87 | |

*DMF: dimethylfuran,
**1,2-HDO: 1,2-hexanediol,
***1,5-HDO: 1,5-hexanediol,
****Others: oligomers + unclear components

Referring to Table 2, as a result of performing the reaction using the catalyst of Example 1 while expanding the temperature range to 120 °C to 200 °C under a constant H₂ pressure at 20 bar, the highest 1,2-HDO yield was obtained under a temperature condition at 130 °C. In addition, as the reaction temperature increased from 130 °C, the reaction time was shortened, whereas the 1,2-HDO yield tended to gradually decrease.

The present disclosure has been described above with reference to the embodiments set forth herein and illustrated in the accompanying drawings. However, these embodiments are merely illustrative, and those skilled in the art will appreciate that various modifications and equivalent embodiments may be made therefrom. Accordingly, the scope of protection of the present disclosure should be defined by the appended claims.

1. A hydrotalcite-based catalyst used for producing 1,2-hexanediol through a reaction between methylfurfural and hydrogen, the catalyst having catalytically active metals supported on a hydrotalcite of Formula 1 below,
[Mg₁₋ₓAlₓ(OH)₂][(CO₃²⁻)_{x/2}]·nH₂O [Formula 1]
(wherein in Formula 1, x is 0.2≤x≤0.33, and n is 0.33≤n≤1),
wherein the catalytically active metals comprise: tin (Sn); and a metal selected from the group consisting of ruthenium and nickel.

2. The catalyst of claim 1, wherein methylfurfural is biomass-derived.

3. The catalyst of claim 1, wherein an amount of tin supported on the catalyst is in a range of 1 to 5 wt %.

4. The catalyst of claim 1, wherein the catalyst is used by reduction without calcination in an oxidizing atmosphere.

5. A method of producing 1,2-hexanediol through a liquid-phase reaction between methylfurfural and hydrogen in the presence of the catalyst of any one of claims 1 to 4, wherein methylfurfural is diluted in any one solvent selected from 1-propanol, isopropanol, 1-butanol, 2-butanol, and a mixture thereof, and subjected to the liquid-phase reaction.

6. The method of claim 5, wherein the reaction is performed at a temperature in a range of 120 °C to 200 °C and at a pressure in a range of 20 to 40 bar.

7. The method of claim 6, wherein the reaction is performed at a temperature in a range of 120 °C to 140 °C and at a pressure in a range of 20 to 30 bar.
